(19) United States
(12) Patent Application Publication
(10) Pub. No.: US 2010/0041983 A9
(43) Pub. Date: Feb. 18, 2010

(54) Device and method for pathology detection

(76) Inventor: Elisha Rabinovitz, Haifa (IL)

Correspondence Address: Pearl Cohen Zedek Latzer, LLP, 1500 Broadway, 12th Floor, New York, NY, 10036, US

(21) Appl. No.: 11/396,916

(22) Filed: Apr. 4, 2006

Related U.S. Application Data
(60) Provisional application No. 60/667,683, filed on Apr. 4, 2005.
(60) Provisional application No. 60/718,751, filed on Sep. 21, 2005.

(51) Int. Cl. A61B 5/05

(52) U.S. Cl. 600/420

(57) ABSTRACT

A method and system may provide detection of pathologies, for example, the detection of cancer in, for example, the gastrointestinal tract utilizing for example magnetically susceptible nano-particles. Ultrasound imaging, MRI technology or other suitable techniques may be used in conjunction to localize detected pathologies.

### RELATED APPLICATION DATA

The present application claims priority from prior provisional application 60/667,683 filed on Apr. 4, 2005, incorporated by reference herein in its entirety.

### FIELD OF THE INVENTION

The present invention relates to the detection of pathologies such as cancer, for example in the gastrointestinal tract.

### BACKGROUND OF THE INVENTION

Early detection of pathologies such as for example cancer or pre-malignant tumors may help to treat the disorder and may reduce related mortalities. Diagnosis may include imaging by, for example, endoscopy, computed tomography (CT), magnetic resonance imaging (MRI), or other suitable imaging systems. Such processes may include if the gastrointestinal tract (GI) is being diagnosed cleaning the colon of residuals of food or other contents and inflating the colon, for example, with air, gas or water. Colorectal endoscopy may involve inserting endoscopes, for example, from the rectum through the colon until the cecum. Complications may occur during such invasive and uncomfortable procedures.

CT or MRI systems may use magnetic or X-ray fields to highlight or detect damaged areas of the body. These systems may be bulky or power costly. For example the weight of an MRI system may reach over 10 tons and consume 125 ampere (480V). The need for high resolution imaging for detection of pathologies as small as few millimeters may necessitate diagnostic tools, such as CT or MRI systems, to be bulky and to consume much power. For example, CT or MRI systems may be room-sized machines. Tests that use these CT or MRI systems may be costly.

MRI imaging relates to the absorption and emission of energy in the radio frequency range of the electromagnetic spectrum, by nuclei having unpaired spins, mainly hydrogen protons. Typically, an MRI scanner operates at 1.5 Tesla in order to achieve a sufficient excitation level of the proton. The hardware components associated with an MRI imager may include a primary magnet, for generating a magnetic field, gradient coils for producing a gradient in the magnetic field, an RF coil, for producing an additional magnetic field or modifying the magnetic field, which may be necessary to rotate the spins by 90 or 180 degrees and may be used for detecting MRI signals, and a workstation, for controlling the components of the MRI imager.

Typically, the magnet is a large horizontal bore superconducting magnet, which provides a homogeneous or substantially constant magnetic field in an internal region within the magnet. A patient may be positioned in the homogeneous field region located in the central air gap for imaging.

A coil system may include gradient coils such as antihelmholtz coils. An antihelmholtz coil may include two parallel ring shaped coils. Current in each of the two coils may flow in opposite directions, which may produce a magnetic field gradient between the two coils. The coil system may include RF coils, which may produce an additional magnetic field or modify the magnetic field, which may rotate the net magnetization in a pulse sequence. Gradient or RF coils may include transmission or reception coils.

Internal imaging, for example, imaging body cavities, may include positioning the patient in a conventional large MRI magnet and using catheters with RF coils. This may produce deficient images because the various orientations of the RF coil. For example, the transverse colon, may not be positioned collinearly with the RF excitation field. This problem has been discussed in U.S. Pat. No. 5,572,132, to Pulyer, et al. entitled, “MRI probe for external imaging”, the disclosure of which is incorporated herein by reference.

Gastrointestinal diagnosis or examination may include a colon clearing preparation procedure and insertion of a probe into the colon with close proximity to the tissue to be examined.

Over the last decade biomedical applications have emerged for nano-particles made of ferromagnetic materials such as iron oxide. A few pure metals like iron, nickel and cobalt maintain their ferromagnetic properties even at nano-size particles. In most cases generating a magnetic field that alternates on a nano-scale may cause particles to exhibit superparamagnetic behavior, magnetizing strongly under an applied field, but retaining no magnetism once the field is removed. Compared to blood cells with a size of a few microns, nano-particles may have diameters ranging from, for example, 300 to 5 nm; other sizes may be used. Ultra-small superparamagnetic particles, for example, ferromagnetic materials with a diameter under 50 micron may penetrate the walls of blood vessels to reach the tissue cells. Utra-small superparamagnetic particles, for example, with diameters ranging from 5 nm to 10 nm, may be sufficiently small to reach intracellular locations.

### SUMMARY

A method and system, according to embodiments of the present invention may provide detection of pathologies and/or target molecules, for example, the detection of cancer in, for example, the gastrointestinal tract utilizing for example magnetically susceptible nano-particles. Ultrasound imaging, MRI technology or other suitable techniques may be used in conjunction to localize detected pathologies.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention.

FIG. 1 is a schematic illustration of a magnetically susceptible nano-particle, in accordance with an embodiment of the present invention;

FIG. 2A, FIG. 2B and FIG. 2C are schematic illustrations of diagnostic systems, in accordance with embodiments of the present invention;

FIG. 3 is a schematic illustration of sensing coils used in a diagnostic system, in accordance with an embodiment of the present invention;

FIG. 4 is a graph that shows the response of the sense coils, in accordance with an embodiment of the present invention; and

FIG. 5A and FIG. 5B are flowcharts of methods for detecting target molecules according to embodiments of the present invention.

It will be appreciated that for simplicity and clarity of illustration, elements shown in the drawings have not necessarily been drawn to scale. For example, the dimensions of some of the elements may be exaggerated relative to other elements for clarity or several physical components included in one functional block or element. Further, where considered appropriate, reference numerals may be repeated among the drawings to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention. Similar reference numerals refer to similar elements of the invention.

Unless specifically stated otherwise, as apparent from the following discussions, it is appreciated that throughout the specification discussions utilizing terms such as “generating,” “computing,” “calculating,” “determining,” or the like, refer to the action and/or processes of a workstation, a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. In addition, the term “plurality” may be used throughout the specification to describe two or more components, devices, elements, parameters and the like.

Embodiments of the present invention provide a method and system for the detection of target substances or pathologies in human and/or animal bodies. For example, tissue diagnosis within the GI tract may be performed that may not require colon cleaning prior to testing. According to one embodiment, a device including for example a magnetic probe, may be operative to perform diagnostic detection by measuring changes or disturbances in magnetic fields made by magnetic particles attracted to target in-vivo substances or particles.

Embodiments of the present invention may provide a method and system for detection of pathologies in human and/or animal bodies. One embodiment may include monitoring relaxation times of particles such as magnetically susceptible nano-particles or water molecules attached to or combined with magnetically susceptible nano-particles. The nano-particles may bond or attach to substances of interest, for example, disordered cells or tissues, or other substances associated with pathologies. An accumulation of magnetically susceptible nano-particles in a patient's body that are bonded to certain cells or tissues may indicate the presence of a pathology. Changes in an induced alternating magnetic field may be monitored to detect accumulated nano-particles.

One embodiment may include monitoring changes in an induced constant or alternating magnetic field due to the presence of water or other molecules attached or adsorbed onto magnetically susceptible nano-particles that may accumulate near disordered cells or tissues and/or bond to target molecules.

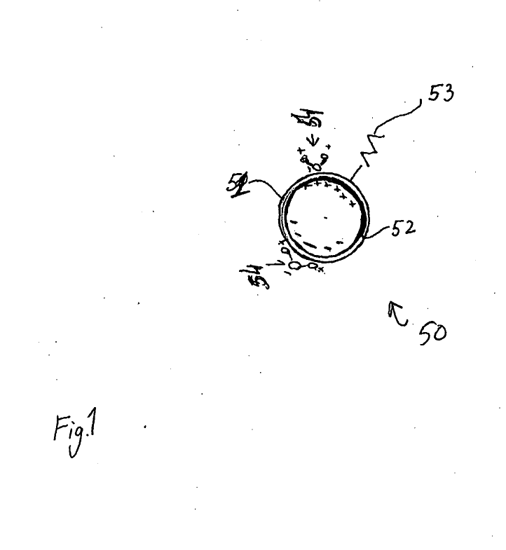

Reference is made to FIG. 1, which schematically illustrates a magnetically susceptible nano-particle, in accordance with an embodiment of the present invention. The detection of substances, antigens or pathologies may involve detecting nano-particles 50 that may bond to, or attach to the substances. Nano-particles 50 have a core 51 which is a nano-particles made of ferromagnetic materials like iron cobalt and any other magnetically susceptible nano-particles with superparamagnetic properties, that may be conjugated with a ligand 53 that may include an antigen, receptor, expression on cell, for example anti-target monoclonal antibodies, or an active part of an antibody, a “hotspot” of an antibody or a protein (e.g., minimal functional domains involved in protein-to-protein interactions and sufficient to induce a biological or chemical response) glycoproteins, glycolipids or “glycocalyx” or bacterial poly-saccharides that can either covalently or non-covalently be attached to specific receptors or antigen or to a antigenic expressed on target sites (e.g., GI tract tissue) optionally: coated with layer 52. Protective layer 52 may coat at least a portion of a surface of nano-particle 50. Protective layer 52 may be, for example, a thin gold layer, to prevent aggregation of the nano-particles in storage or during administration into blood. Other protective layers may be used. In one embodiment, protective layer 52 may be used to reduce toxicity. In another embodiment protective layer 52 may provide a surface for attaching one or more receptor molecules 53 (e.g. antibodies) to nano-particles 50. Once in the body, water molecules 54 from the biological environment may adsorb onto, attach or coat the surface of the magnetically susceptible nano-particles 50.

In one embodiments of the invention the nano-particles may be introduced by swallowing. In one example, conjugated nano-particles may for example be encapsulated in a pill and released only when the relevant organ of the GI tract may have been reached. For example the conjugated nano-particles may be, for example controllably released in the colon e.g. the nano-particles are contained in a pill that may be coated with a pH sensitive coating that may degraded at pH greater than 7.

According to one embodiment, once the pill may be administered to a subject and once it may reach the colon the pill or other housing or container may decompose and release the nano-particles. A high concentration of nano-particles may be attached to the cancerous tumor with the targeted antibody.

In yet another embodiment a syringe or an intravenous injection or any other suitable delivery mechanisms may be used to administer nano-particles 50, for example, into the blood stream.

In one embodiment, once nano-particles 50 may be introduced into a body they may diffuse in the blood stream or other lumen. If pathologies exist that express antigens, such as polyps or cancerous tumors, nano-particles 50 may react with the antigen or targeted agent and accumulate in or in close vicinity to the disorder.

According to one embodiment, nano-particles 50 may be introduced to the body being examined, for example, by injecting the subject with a suspension of nano-particles 50. Some time later, such as a few minutes later, for example, when at least some nano-particles 50 have had sufficient time to travel through the blood stream where they may bond or attach to antigens present in the body, the body may be screened.

In one embodiment nano-particles 50 may include paramagnetic nano-particles 51. Unlike superparamagnetic particles, paramagnetic particles typically retain their magnetization once an external magnetic field is withdrawn. Nano-particles 51 may be made from magnetically susceptible substances, for example, pure transition metals, such as Fe, Ni and Co. Paramagnetic nano-particles typically have a larger magnetic moment than superparamagnetic nano-particles of similar size. Thus, in an alternating magnetic field, the field distortion due to the accumulation of paramagnetic nano-particles is typically larger than super-paramagnetic particles of similar size.

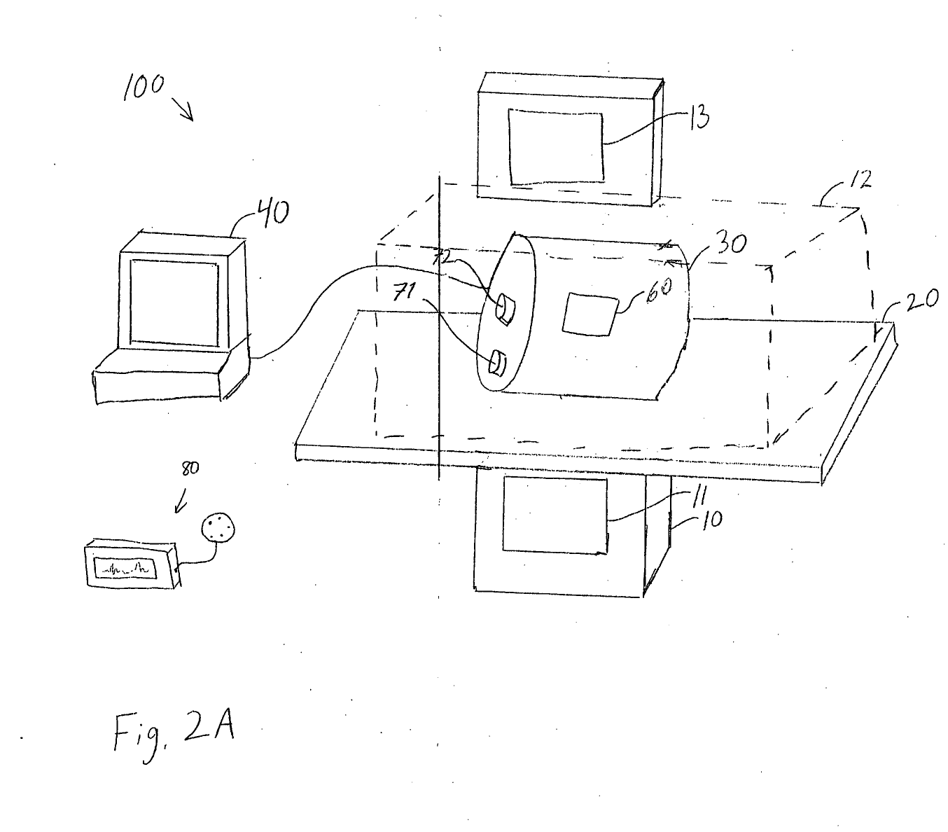

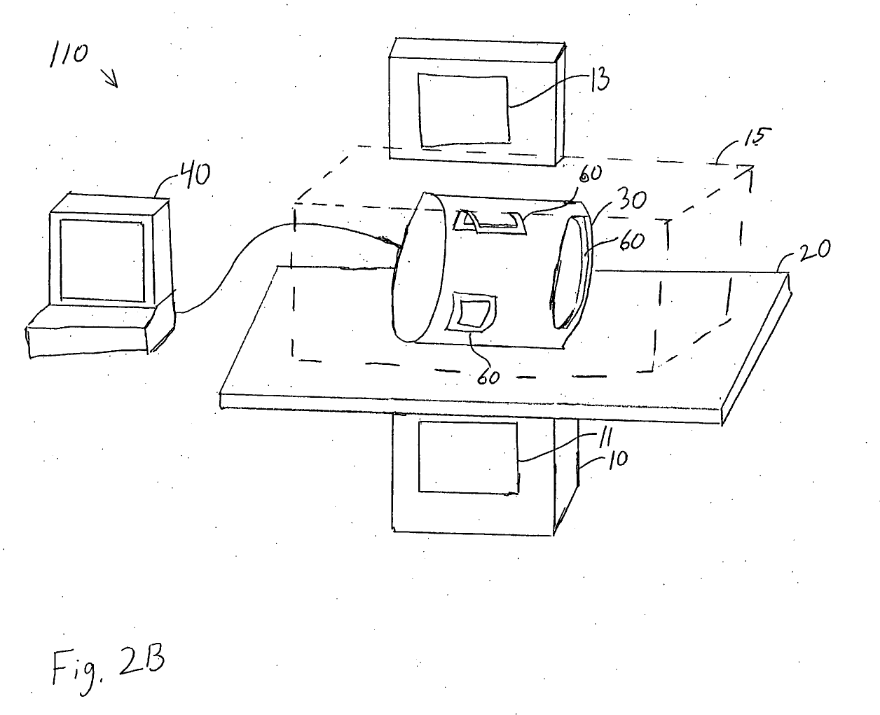

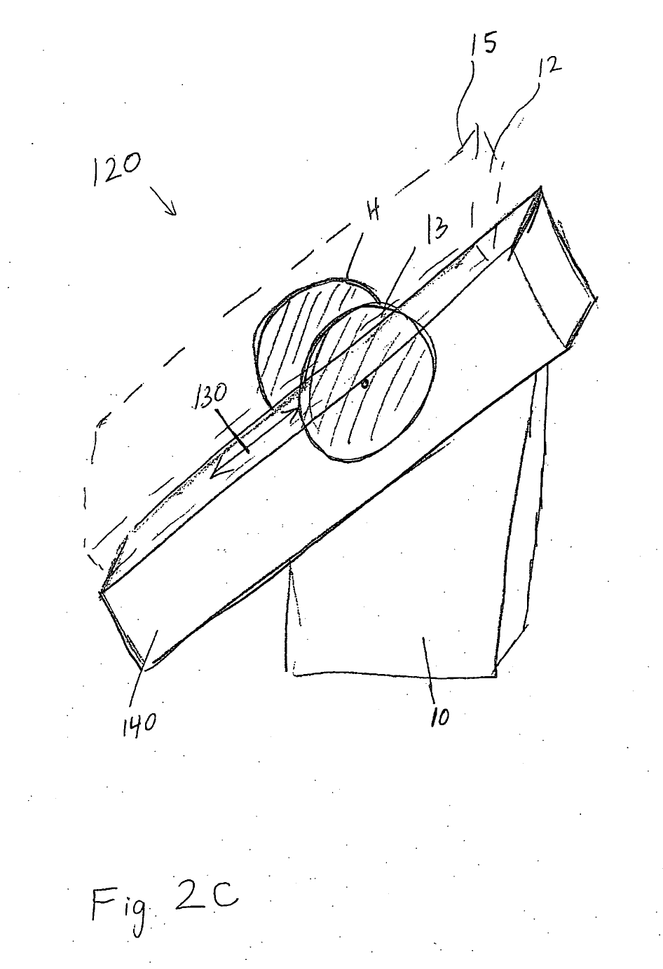

Reference is made to FIG. 2A, FIG. 2B and FIG. 2C, which schematically illustrate diagnostic systems, and the operation thereof, in accordance with an embodiment of the present invention. In one embodiment, system 100 may detect magnetically susceptible nano-particles 50, for example, as described in FIG. 1, for example, with receptor molecules 53 that may include antibodies that may typically attach or bond to substances of interest, for example, pathological or disordered biological structures. Systems 110 and/or 120 may detect water molecules 54 that attach, adsorb or are attracted and/or bond to nano-particles 50.

Systems 100, 110 and 120 may include a table or bed 20, a belt 30, one or more magnets 11 and/or 13, one or more coils 60, a base 10, a workstation 40 and an imaging and/or location probe 80, e.g. an ultrasound probe 80. Bed 20 may support a patient's body. Belt 30 may be cylindrically shaped and may be positioned on bed 20 that may be closed around the body of a subject, such that the body may be positioned substantially within the range of the generated alternating magnetic field 12. Other suitable components may be used. Systems 100, 110 and 120 may generate a magnetic field 12 which may be for example alternating or constant. Different elements of systems 100, 110 and 120 may be supported by base 10.

According to one embodiment a workstation 40, for example, a computer or computing system, may be used for controlling the components of systems 100, 110 or 120 and presenting the resulting detection data and/or image data. Workstation 40 may include a data processor that may analyze the data received and may be in communication with storage e.g., transferring data to and from storage units. The data processor may provide the analyzed data to monitor 18, where a user (e.g., a physician) may view or otherwise use the data. In some embodiments, data processor and/or workstation 40 may be configured and/or may be implemented using a hand-held device.

Monitor 18 for example may additionally be used to display one or more images or a stream of images e.g., images of the GI tract or of other imaged body lumen or cavity as provided from an MRI or from ultrasound or x-ray machine and may display the data relating to the location or position data of the concentration of the covalently bonded nano-particles 50, which may indicate a location of a pathology. In some embodiments, for example, both an image, and its position or location (e.g., relative to the body lumen being imaged) may be presented and the position of bonded nano-particles 50 or water molecules 54 bonded to nano-particles 50, indicating a substance indicating a pathology, may be superimposed on those images.

Referring to FIG. 2A showing a system 100 that may be for example a magnetometer system that may be capable of inducing an alternating magnetic field and measuring changes in the induced magnetic field. In one embodiment, system 100 may only detect nano-particles 50 that are attached or bonded to biological structures, through for example ligand 53.

According to embodiments of the present invention, components of system 100 may provide or generate an alternating magnetic field 12, across portions of the body being examined. Typically the alternating magnetic field 12 may cause nano-particles 50 to vibrate, move or rotate.

Dipole moments of magnetically susceptible nano-particles 50 may spontaneously rotate to align toward a field direction. The anisotropy energy barrier of the particle, E, which is proportional to its volume, may inhibit the dipole moment from rotating, but may be overcome with sufficient thermal energy, k_{B}T, where T is the temperature and k_{B} is the Boltzmann's constant. Thus, Néel relaxation occurs on a time scale ?_{N}=?₀e^{e/k}_{B}^{T}, which is exponentially proportional to the volume of nano-particle 50. In addition to Néel relaxation, nano-particles in suspension may undergo Brownian rotation, which may randomize the orientation of the dipole moments. These fluctuations may occur on a time scale, ?_{B}, which may depend linearly on nano-particle 50, for example, a 20-nm magnetite nano-particle 50, for an ideal ?_{N}˜1 s and ?_{B}˜1 μs. The time for the effective relaxation process may be the faster of the two times, ?_{N} and ?_{B}. As a result, when the field is turned off, the free or unbound magnetically susceptible nano-particles 50 may randomize by Brownian rotation in a few microseconds. In contrast, the bound magnetically susceptible nano-particles 50 typically do not rotate and relax by the Néel mechanism for a period of time, for example, several second, after the field is turned off. Thus, magnetically susceptible nano-particles 50 may produce measurable discrepancies in the generated alternating magnetic field 12 for the period of time. As a result, substantially slow responding magnetic probe 60 may detect substantially only decaying magnetic fields 12 discrepancies produced by bound magnetically susceptible nano-particles 50.

System 100 may include various components and configurations of components that may be used to generate an alternating magnetic field 12 created for example by magnets 11 and 13 and measure discrepancies in the alternating magnetic field 12. In one embodiment alternating magnetic field 12 may be of a frequency in a range to measure only those magnetically susceptible nano-particles 50 that are bonded to a substance or target molecule indicating a pathology. The frequency range may depend on particle size and the bonding energy between particles and the target molecule or particle.

In some embodiments, since these bonded magnetic nano-particles 50 typically do not substantially orient themselves with the alternating magnetic field, they may cause a disturbance in the magnetic field. Magnetic probe 60, for example, a coil vibrating magnetometer, hall-effect magnetometer or GMT based magnetometer may be used to measure disturbances in the applied, alternating magnetic field at different locations throughout a body. Such disturbances may indicate the presence of or an accumulation of attached or bonded nano-particles 50 with ligand 53, and thus the presence of the corresponding antigens, pathologies or substance indicating a pathology.

Magnets 11 and/or 13 (e.g., constant or alternating magnets) of system 100 may produce alternating magnetic field 12.

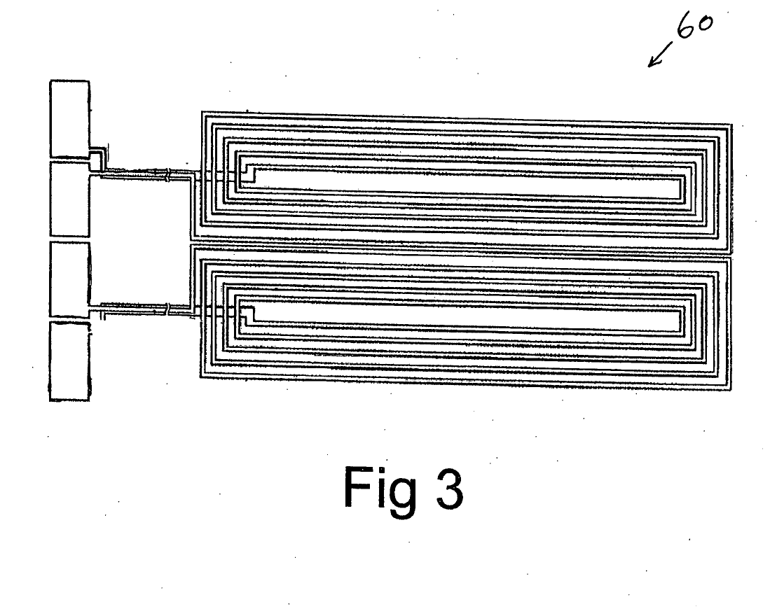

Reference is made to FIG. 3, which schematically illustrates the magnetometer 60 based on spiral coils used in diagnostic system 100, in accordance with an embodiment of the present invention. In one embodiment, system 100, described with reference to FIG. 2A, may include spiral coils 60. Spiral coils 60 may be arranged in a symmetrical gradiometer configuration and installed in for example pairs with symmetry to the short axis of bed 20. Other numbers of coils and other positions may be used. For example, coil 60 may include two spiraled coils that may be arranged in perpendicular configurations. In the absence of local disruptions in generated magnetic fields 12, coil 60 may produce current in equal and opposite directions. Thus the current contribution from each of the two spiraled coils may cancel and the overall current of coil 60 may be zero. Coil 60 (e.g., contained in belt 30) may scan magnetic fields 12 along the patient's body and pass over an area that contains bonded nano-particles 50 that produce a local discrepancy in magnetic fields 12. Typically, only one of the two spiraled coils may pass over such areas at a time. If there is a discrepancy in the magnetic field of the one spiraled coil, there will be a discrepancy in the current of that spiraled coil. Since the other spiraled coil typically does not pass over the area with the local discrepancy in magnetic fields 12 the coil will not have discrepancies in its current. Thus, the two spiraled coils may have different currents and the overall current of coil 60 will not be zero.

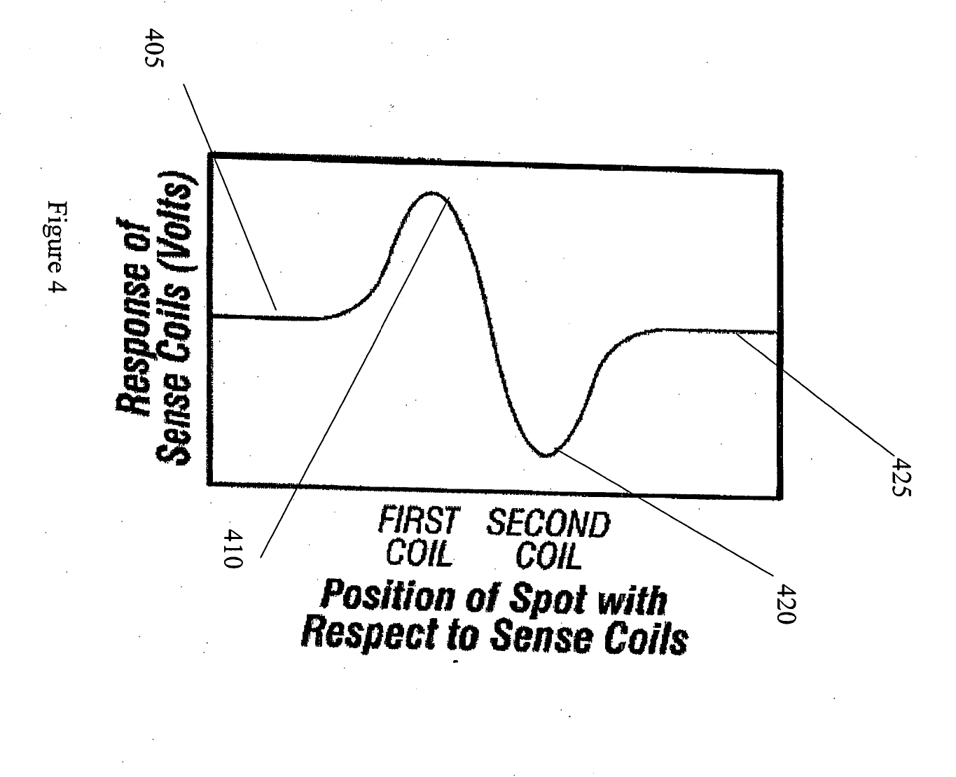

Reference is made to FIG. 4, which schematically illustrates a graph that shows the response of a spiral coil against the position of the spiral coil according to an embodiment of the present invention. The response of spiral coil 60 (FIG. 3) may be for example measured in volts or other suitable units. Curve 405 may indicate the electric current of coil 60 as it passes over an area of the body with no local discrepancies in magnetic fields 12. The current contribution from each of the two spiraled coils may cancel and the overall current of coil 60 may be zero. Curve 410 may indicate the current of coil 60 as a first spiraled coil of coil 60 passes over an area of the body with a local discrepancy in magnetic fields 12. The two spiraled coils have different currents and the overall current of coil 60 will not be zero. Since the two spiraled coil are attached and scan substantially the same areas, after one of the spiraled coil passes over an area with a local discrepancy in magnetic fields 12, so will the other spiraled coil. Curve 420 may indicate the current of coil 60 as the other or second spiraled coil of coil 60 passes over the same area with a local discrepancy in magnetic fields 12. Due to the inverse or mirrored configuration of the second spiraled coil with respect to the first, discrepancies in its current of the second spiraled coil may be of equal amplitude and opposite direction, as indicated by curve 420. Curve 425 may indicate the current of coil 60 in a position away from local discrepancies in magnetic fields 12. In such a position, there is typically no substantial discrepancies in the current of either of the spiraled coils and the overall current of coil 60 may return to zero.

Referring again to FIG. 2A, in other embodiments other magnetometers are possible e.g. hall-effect or GMR devices arranged in a Wheatstone bridge arrangement may scan magnetic fields 12 along the patient's body and pass over an area that contains bonded nano-particles 50 that produce a local discrepancy in magnetic fields 12. Typically, if there is a discrepancy in the magnetic field of the one branch of the bridge, there will be a discrepancy in the current of the bridge and the overall current of will not be zero.

Referring to FIG. 2A, in one embodiment of the present invention, system 100 may include additional localization devices. Localization devices 80 for example may include a hand held ultrasound traducing system. In yet other embodiment transducers 72, for example, piezoelectric transducers, that may be arranged in direct contact with a patient's body. In one embodiment, a signal sent by transducer 72 may reflect, for example, off the walls of the patient's body, for example, near the gastrointestinal tract, and may be received by transducers 72. In one embodiment, a minimum of three transducers 72 may be used. A plurality of transducers 72 may be arranged in belt 30 separated by a predetermined distance. Cross correlating signals from transducers 72 may be processed, for example, by workstation 40 to produce localization data relating to magnetic field measurements of the examined body.

In another embodiment, multiple transducers 72 may operate in combination, where at least three receivers 71 may be used. In one embodiment, receivers may function as transducers. The receivers 71 may be arranged at different locations, for example, in direct contact with a body. A signal, sent by transducer 72 may be received by receiver 71, and may then be sent to workstation 40. Localization data relating to magnetic field measurements of the examined body may be produced based on the distance between transducers 72 and receivers 71 and the differences in the amplitudes of the signals receivers 71 receive.

Referring to FIG. 2B showing system 110 that may be an MRI based system, e.g. a low power MRI system and/or system based on MRI technology. System 110 may be used to detect pathologies in human and/or animal bodies by detecting resonance effect in water molecules 54 that may be attracted, or adsorbed onto the surface of magnetically susceptible nano-particles 50 that bond to target substances. The presence of an accumulation of such adsorbed water molecules 54 may indicate a presence of the target substances.

After administration of a pulse of RF radiation, the excited water molecules may start to relax due to two main processes: intra-molecular interactions between protons and associated electrons in the same molecule and intermolecular interactions due to diffusion and molecular rotation. In pure or unbound water those interactions may be weak and relaxation time may be relatively long, e.g., about 4 seconds. Water molecule adsorbed to magnetically susceptible nano-particles 50 may still rotate under the effect of RF energy as a result of Brownian rotation of the particle itself Since nano-particles 50 are typically small, the effect of unbounded magnetically susceptible nano-particles 50 on the relaxation time may be relatively small. In contrast, water molecules 54 adsorbed to the particle surface that is covalently bonded typically are not free to rotate and may relax only by the Néel mechanism of the particles itself. Thus, its relaxation time is very short.

In some embodiments of the invention, intermediate ranges of relaxation times may also be detected, for example, relaxation times that correspond neither to bound water molecules 54 nor free water molecules. For example, magnetically susceptible nano-particles 50 may get into a cell or macrophage. The nano-particles' 50 ability to move is limited which may lead to an intermediate range of relaxation times. In some embodiments the size of magnetically susceptible nano-particles 50 may be chosen according to the desired relaxation time.

Referring to FIG. 2B, system 110 may generate a constant magnetic field 15, for example with magnets 11 and 13. According to one embodiment system 110 may include one or more components to excite water molecules, for example, RF coils 60 that may generate RF pulses in the presence of magnetic field 15, which may for example, resonate water molecules 54.

Adsorbed water molecules 54 typically have shorter relaxation times than free, unadsorbed or unbound water molecules. Thus, by detecting water molecules 54 bound to nano-particles 50 that may bond to substances indicating a pathology, system 110 may detect the pathological structures. The relaxation time or behavior of water molecules may be measured, for example, using low intensity MRI devices with modified hardware and software to analyze relaxation time of each Vauxhall

In one embodiment, RF coils 60 may emit RF pulses that in the presence of constant magnetic field 15 may resonate water molecules throughout the body. When the RF Eco pulse is turned off, the excited molecules typically relax or realign with the conformation and/or orientation of the nano-particles. Typically the relaxation time of water molecules 54 that are adsorbed to a covalently bonded particle is faster than the relaxation time of water molecules that are not bonded. Embodiments of the present invention may use the difference in the relaxation behavior of water molecules 54 bonded to nano-particles 50, bonded to other particles and not bonded, to detect or isolate water molecules 54 bonded to nano-particles 50.

Introducing magnetically susceptible nano-particles 50 into a patient's body, for example, may significantly affect the relaxation time and behavior of water molecules in the body, for example, due to local magnetic fields created by the magnetically susceptible nano-particles 50. In the presence of an introduced magnetic field 15 preferably with a main power field of 0.04-0.12 Tesla), an RF pulse may be emitted (e.g. by RF coils 60) at the resonance frequency of the water molecules 54.

System 110 may include additional coils 60 and additional for example, gradient coils, may create a gradient magnetic field 15 along the Z-axis, for example, perpendicular to the surface of bed 20. There may be a magnetic amplitude and direction associated with each position along a gradient magnetic field 15. The amplitude of the gradient magnetic field may allow nano-particle MRI imaging. The direction of the gradient magnetic field 15 associated with the amplitude of gradient magnetic field 15 may provide localization data for those nano-particles 50. Workstation 40 may process and present data to a viewer, for example, magnetic field measurements, images or image data, etc.

Referring to FIG. 2C, in one embodiment of the present invention, system 120 may include a bed or platform 140, which may be used to position the patient System 120 may include methods, components and/or configurations used in systems 100 and/or 110 for example either an MRI based system and/or a magnetometer based system. For example, system 120 may generate a constant magnetic field 15 and/or an alternating magnetic field 12. System 120 may produce and magnetic fields and measure discrepancies in those fields, according to embodiments described with reference to FIG. 2A and FIG. 2B. Bed 140 may move and/or rotate in order to refine or change the body's location during the diagnostic test period. Bed 140 may move relative to a device that generates or scans magnetic fields 12 or 15 (e.g., measures magnetic field discrepancies), which may include magnets 11 and/or 13 and coils 60. The location of discrepancies in magnetic fields, and thus of nano-particles 50 or water molecules 54 bonded to nano-particles 50 that produce the discrepancies, may be determined by the location of the magnetic field scanning device relative to bed 140 or the body at the time of detection.

Other suitable configurations may be used. For example, in an alternate embodiment, magnet 11 may be positioned above bed 140 and may move along an upper track, magnet 11 may be located in or adjacent to base 10 and bed 140 may be moved on base 10 to allow magnetic field 12 or 15 to surround the patient's body.

In some embodiments of the present invention, systems 110 and 120 may generate low intensity constant magnetic fields 15, for example, with intensities of 0.04-0.2 Tesla. Typically, such low intensity constant magnetic fields 15 produce poor quality MRI images of body tissues, and thus, are not used in conventional MRI procedures. The low intensity constant magnetic field 15 and/or low detection times may enable detection of particles, for example, water molecules 54 (bound or unbound) and their location relative to a given space.

In one embodiment, systems 100, 110 or 120 may include an additional imaging or detection device or mechanism that may be used to scan, map or image the patient's body, for example, organs and/or tissues. For example, systems 100, 110 or 120 may include an imaging device that may include an imager and a localization tool, which may provide information relating to location, for example, of a substance indicating a pathology, the imaging device or an element thereof, magnetically susceptible nano-particles 50, water molecules 54 bonded or adsorbed onto magnetically susceptible nano-particles 50, anatomical structures and/or other related objects or devices. Additional imaging mechanisms may include, for example, ultrasound screening, MRI, X ray screening, CT, or any other suitable method.

The results of the magnetic field measurement may be combined with additional image data to provide both detection and localization of the antigens or pathologies. Systems 100, 110 and 120 may localize detected substances or pathologies with respect to components of the systems 100, 110 and 120 (e.g., bed 20 or 140) or with respect to another reference such as a position along a gradient magnetic field 15 (e.g., which may also be associated with the positions of components of the systems 100, 110 and 120). Image data may provide localization of organs with respect to the positions of components of the systems 100, 110 and 120. Thus, by combining image data and detection data, systems 100, 110 and 120 may provide both detection and localization of the antigens or pathologies. For example, data from the low intensity magnetic probe 80 and the additional imaging results may be combined to localize detected magnetically susceptible nano-particles 50 to a specific body part, such as an organ or tissue, thus providing detection of, for example, a pathology location. Combining data that may be collected by separate mechanisms may involve normalizing a coordinate systems and/or superimposing data and/or results as is known. For example, workstation 40 may combine data. In one embodiment, at each location in a body, recorded by an imager, systems 100, 110 or 120 may provide data relating to antigens or the concentrations of substances indicating a pathology. For example, systems 100, 110 or 120 may provide data relating to concentrations of magnetically susceptible nano-particles 50, water molecules 54 bonded or adsorbed onto magnetically susceptible nano-particles 50, the decay of such particle concentrations and/or other pathology detection data.

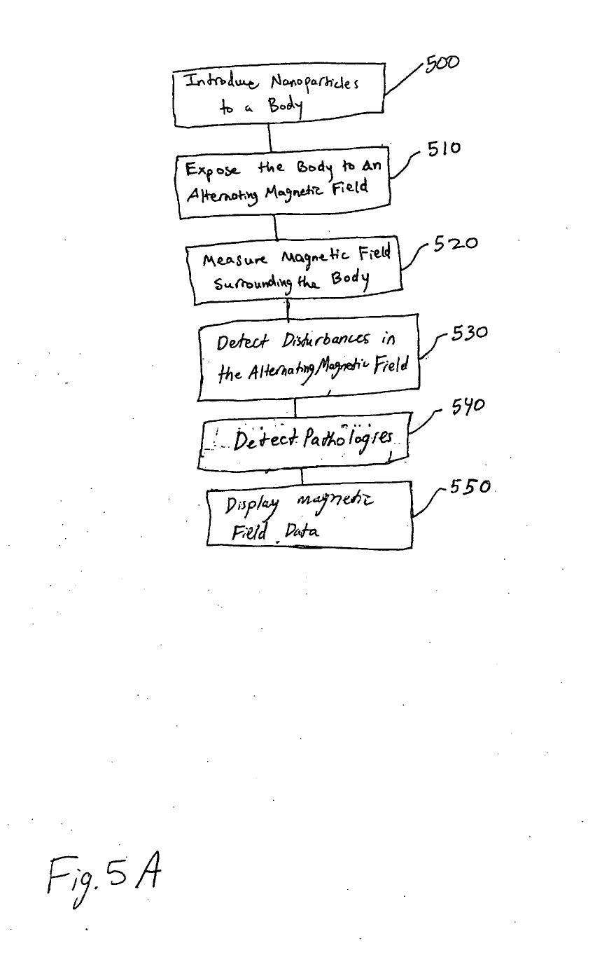

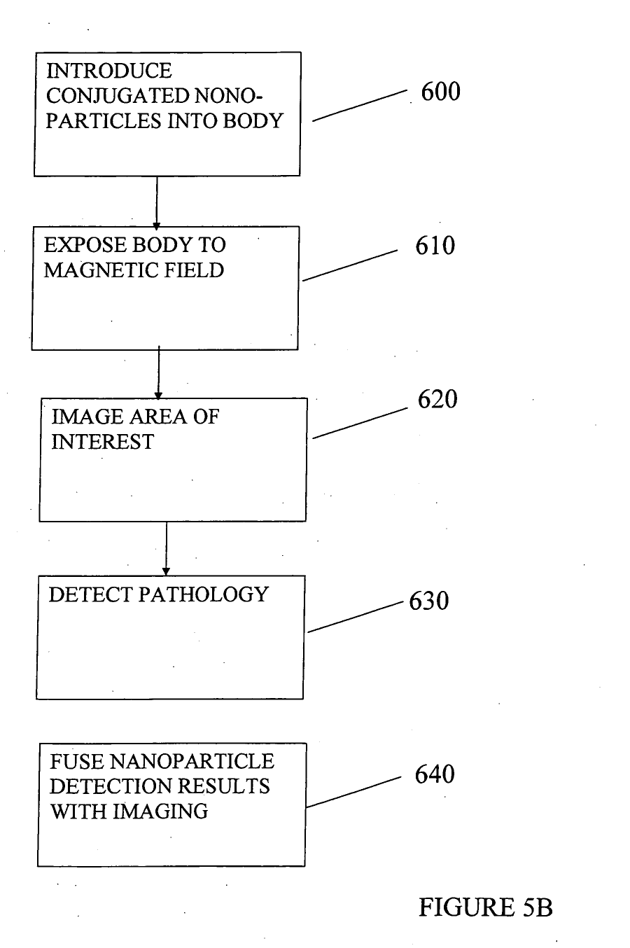

Reference is made to FIG. 5A, which is a flowchart of a method for detecting target substances according to one embodiment of the present invention.

In operation 500, magnetically susceptible nano-particles may be introduced into a patient's body by, for example, injecting or swallowing nano-particles (e.g., in suspension). The nano-particles may be conjugated or coated with one or more receptor molecules. For example, receptor molecules may include antibodies, which are attracted to and may typically accumulate near a target substance. The conjugated nano-particles, via the receptor molecules, may be more concentrated near corresponding antigens than throughout a body.

In operation 510, the body may be exposed to an alternating magnetic field. The alternating magnetic field around the body may excite the magnetic nano-particles inside the body. In one embodiment, operation 510 may be executed some time after operation 500, for example, a few minutes after, so that at least some of the introduced nano-particles have substantially sufficient time to diffuse or travel through the blood stream where they may accumulate near a target substance.

In operation 520, disturbances in the alternating magnetic field may be detected. In one embodiment, one or more devices may be used, for example, portable magnetic probe sensors such as magnetometers. The device may scan the body or the area substantially surrounding the body and may generate data relating to the magnetic field, for example, over a period of time, of the body or of the surrounding area. The device may process data relating to the magnetic field surrounding the body. In one embodiment, a magnetometer may substantially only detect disturbances in the alternating magnetic field surrounding the body.

The nano-particles introduced in operation 500 that have bonded or attached to a biological substance typically do not align or align less with the produced alternating magnetic field. Since these bonded or attached magnetically susceptible nano-particles typically do not substantially align with the produced alternating magnetic field, they cause disturbances in the alternating magnetic field that is detected by the magnetometer indicating a pathology.

In operation 530, a localization device may localize discrepancies in the magnetic field for example detected in operation 520, for example, by an array of transducers and receivers creating an ultrasound image, or an hand held ultrasound or by by MRI images. The detection data may be localized with respect to system position. Additional image data may provide the location of organs or biological structured with respect to system position. The detection data may be combined with the image, for example, to provide both detection and localization of the pathologies.

An imaging device may generate image data that relates to the body. Magnetic field detection measurement may be combined with image data to provide both detection and localization of the antigens or pathologies.

In operation 540, the fused data of the disorder location and image location may be displayed. The monitor may be part of a workstation that a healthcare professional may view. In one embodiment, the monitor may display data relating to the magnetic field surrounding the body, disturbances in that field, localization data, image data or any combination, derivation or superimposition of these data.

Other operations or series of operations may be used.

Reference is made to FIG. 5B, which is a flowchart of a method for detecting target substances according to one embodiment of the present invention.

In operation 600, magnetically susceptible nano-particles may be introduced into a body. Due to the magnetic properties of the magnetically susceptible nano-particles and water molecules, once the nano-particles are introduced into the body, water molecules may attach or adsorb onto the surface of the nano-particles.

In operation 610, the body may be exposed to a magnetic field. The magnetic field may include a constant or alternating magnetic field. In addition, electromagnetic radiation such as, for example, a series of RF pulses or signals may be applied and the response may be detected and measured. The patient's body may be positioned within the range of the magnetic field. The RF pulses may be substantially at the resonance frequency of water molecules. Unbound water molecules may resonate with relatively long relaxation times. Bound water molecules, typically may have short relaxation times, which may depend on the particles to which the water molecule may bond and/or the type of bond. Water molecules substantially bonded or adsorbed onto the nano-particles may have a specific relaxation time, which may depend on the size and/or concentration of the nano-particles.

In one embodiment, operation 610 may be executed some time after operation 600, for example, a few minutes after, so that at least some of the introduced nano-particles have substantially sufficient time to adsorb water molecules and to travel through the blood stream where they may accumulate and bond or attach, for example, around antigens or pathologies.

In operation 620, in one embodiment, imaging (e.g., MRI using RF and gradient coils or ultrasound may be used). In operation 630, pathologies may be detected.

The results of the magnetic field detection measurement may be combined with additional image data, for example, recorded by an imaging device, to provide both detection and localization of the antigens or pathologies. In operation 640, the fused data may be displayed. Other operations or series of operations may be used.

It will be appreciated by persons skilled in the art that the present invention is not limited to what has been particularly shown and described hereinabove. Rather the scope of the present invention is defined only by the claims, which follow:

1. A method comprising:
introducing magnetically susceptible nano-particles into a body;
exposing the body to an alternating magnetic field;
detecting disturbances in the magnetic field surrounding the body; and
detecting concentrations of the nano-particles.

2. The method of claim 1, wherein detecting a concentration of the nano-particles indicates the presence of substances indicating a pathology.

3. The method of claim 1, wherein the nano-particles are conjugated with receptor molecules that are attracted to substances indicating a pathology.

4. The method of claim 1, wherein the nano-particles comprise a protective layer.

5. The method of claim 1, wherein the nano-particles comprise ligand.

6. The method of claim 1 comprising generating data on a location of the nano-particles.

7. A method comprising:
introducing magnetically conjugated susceptible nano-particles into a body;
exposing the body to a magnetic field;
resonating water molecules;
detecting water molecules that are attached to the nano-particles.

8. The method of claim 7, wherein unbound water molecules are resonated by radio frequency pulses.

9. The method of claim 7, wherein detecting the water molecules indicates the presence of substances indicating a pathology.

10. The method of claim 7, wherein the nano-particles are conjugated with receptor molecules that are attracted to the substances indicating a pathology.

11. The method of claim 7, wherein the nano-particles comprise antibodies.

12. The method of claim 7 comprising generating data on a location of the water molecules that are attached to the nano-particles.

13. A system comprising:
a plurality of magnetic particles, the particles comprising a substance attracted to a target in-vivo substance;
a device to generate an alternating magnetic field; and
a device to measure disturbances in magnetic fields.

14. The system of claim 13, wherein the substance comprises receptor molecules that are attracted to substances indicating a pathology.

15. The system of claim 13, wherein receptor molecules comprise antibodies.

16. The system of claim 13 comprising a processor to detect a concentration of the nano-particles.

17. The system of claim 13, wherein the device to measure magnetic fields is to detect disturbances in the substantially alternating magnetic field surrounding the body.

18. A system comprising:
a plurality of magnetic particles, the particles comprising a substance attracted to a target in-vivo substance;
a device to generate a magnetic field;
a device to generate radio frequency pulses at the resonance frequency of unbound water molecules; and
a device to measure disturbances in magnetic field.

19. The system of claim 18, wherein disturbances in magnetic field are caused by water molecules that are attached to the substance.

20. The system of claim 18, wherein the substance comprises receptor molecules that are attracted to the substances indicating a pathology.

21. The system of claim 18, wherein the substance comprises antibodies.

22. The system of claim 18 comprising a processor to detect a concentration of the substance.
